(19) United States
(12) Patent Application Publication
(10) Pub. No.: US 2003/0125792 A1
(43) Pub. Date: Jul. 3, 2003

(54) Exterior stent and its use

(76) Inventors: Sidney Braginsky, Dix Hills, NY (US); Russ A. Houser, Livermore, CA (US)

Correspondence Address: Cohen & Grigsby, P.C., 15th Floor, 11 Stanwix Street, Pittsburgh, PA, 15222, US

(21) Appl. No.: 10/335,680

(22) Filed: Jan. 2, 2003

Related U.S. Application Data
(63) Continuation-in-part of application No. 10/037,817, filed on Jan. 3, 2002.

(51) Int. Cl. A61F 2/06

(52) U.S. Cl. 623/1.11; 623/1.13; 623/1.15

(57) ABSTRACT

A support apparatus and its use is provided comprising a substantially support layer with an inside surface and an outside surface; and a securing element for securing the support layer to the exterior of a body lumen. The method for implanting the support to the exterior of a body lumen comprises providing for an exterior support, inserting the stent around a desired location on the exterior of the lumen, and providing for controlled expansion of the lumen such that it contacts the support sufficiently to secure it to the lumen.

### CROSS REFERENCES TO RELATED APPLICATIONS

This application claims the benefit of U.S. Nonprovisional application Ser. No. 10/037,817 filed on Jan. 3, 2002.

### BACKGROUND OF THE INVENTION

Intralumenal grafting and stents are used to hold a body lumen open and enlarged. This involves the percutaneous insertion and placement by catheter of a cylindrical prosthetic device within a body lumen. Stents are used in the vascular system, respiratory, biliary and urinary tracts. Typically, they are composed of stainless steal springs, wire in a zigzag pattern or helically wound springs.

Intralumenal grafts create several potentially dangerous conditions. If the grafts are under expanded at their target location in the lumen or under sized for the lumen, they do not secure themselves properly and can migrate away from the location. An over expanded or oversized graft can rupture the lumen.

### SUMMARY OF THE INVENTION

The present invention is directed to a method and prosthesis that is capable of opening and enlarging body lumens from the exterior. The invention provides for external, atraumatic vessel support. The support, which can be interchangeably referred to as a stent, may be made from a biologically inert material and may be reinforced with a layer consisting of a polymer or metallic braid, wrap or other pattern. It may be secured to the exterior of the lumen using an adhesive, protruding member, suture or a combination of these means.

The result of the use of the present invention is a larger and reinforced lumenal vessel diameter. One effect of increased lumenal diameter is an increase in blood flow for cardiovascular vessels.

It is an object of the present invention to provide an atraumatic exterior lumenal stent.

It is another object of the present invention to provide an atraumatic exterior lumenal stent that enlarges lumenal diameters.

It is still another object of the present invention to provide a method for enlarging the diameter of body lumens using an exterior lumenal stent device.

It is still another object of the present invention to provide a method and device to increase lumenal diameter without implanting a foreign object into the body lumen, thereby reducing the risk of lumenal rupture and incidences of acute thrombosis and intimal hyperplasia.

### BRIEF DESCRIPTION OF THE DRAWINGS

FIG. 1 is a schematic depiction of the a two-piece stent with a hinge.

FIG. 2 is a depiction of a bifurcated stent without seam detail.

FIG. 3 is a depiction of the cross-section of an embodiment of a one-piece stent.

FIG. 4 is a schematic depiction of a body lumen that is a candidate for the presently claimed process and device.

FIGS. 5 and 6 depict an embodiment of the claimed process of providing exterior lumenal in a lumen having a lesion.

FIG. 7 depicts a lumen having had the presently claimed device applied and the presently claimed method performed thereon.

### DETAILED DESCRIPTION OF THE INVENTION

This stent may comprise a single or multiple pieces. In FIG. 1 the multi-piece piece design would be constructed as two semi-cylindrical elements that are integrally connected at a hinge or flexible element. The two semi-cylindrical elements 10 are constructed to connect to each other opposite the hinge. The ends opposite of the hinge or flexible element are fastened together by a latch or other conventional means that will hold the device in a cylindrical shape.

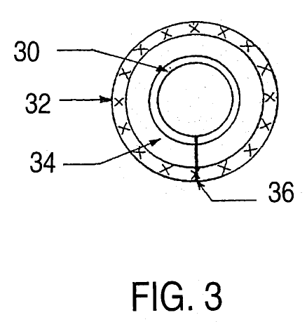

FIG. 3 shows a cross-section of an embodiment of the invention, wherein the stent is a single piece. A seam 36 runs the width of the cross-sectional area. It will be appreciated that the multi-piece design can posses the same cross-sectional features as said multi-piece design may be comprised of the same layered structure.

In an embodiment, the stent is has multiple layers and an outer layer 32 for reinforcement. In this embodiment, a support layer is an interior layer 34 and may be made from a soft, compliant, atraurnatic material. For example, the interior layer may be made from ePTFE, woven Dacron, or other compliant material. Alternate embodiments may or may not contain the outer reinforcement layer. In said alternate embodiments, the support layer 34, which in this embodiment is equivalent to the interior layer of the embodiment having the outer reinforcement layer, may be made from expanded PTFE (ePtfe), woven Dacron or other suitable material. Alternatively, it may be made from biodegradable material, including but not limited to collagen, synthetic polymers such as polyglycolic acids, polylactids, polyhydroxybutyrates, polyhydroxyvaleriates, polydioxanons, modified starches, gelatins, modified celluloses, polyglycols, polyacrylic acids, polymethacrylic acids, natural or synthetic aliphatic or hydroxy polymer of lactic acid, glycolic acid or some combination of these or others. In an embodiment, the outer layer 32 is provided and is stronger and less compliant than the inner layer. In this embodiment, the outer layer is comprised of a polymer or metallic braid, wrap or other pattern. The whole stent may also be flexible.

On the inner diameter of the support layer is a securing element 30. In an embodiment, an inner diameter of the interior layer may be coated or comprised of a biologically inert adhesive, which acts as the securing element. The adhesive is used to adhere to the exterior of the target body lumen. The stent may be secured to the lumen wall by a securing element such as an adhesive 30 such as cyanoacrylate, fibrin, fibrinogen, or some combination of these. However the adhesive is not limited to these materials. The adhesive could have a surface contact cure method such as UV light, heat or some combination. Alternatively, the stent may be secured by alternative securing means such as penetrating barbs or hooks, or reversible adhesive bonding between the lumen and stent. Additionally, conventional locking tabs or a ratchet system may be used to tighten and secure the stent onto the lumen.

The device is constructed in different sizes to be able to approximate the size of the target lumen or is able to expand or contract to properly reach the lumen size.

Extrusion, molding, dip coating or a combination of these methods or other methods may be used to manufacture the stent. The stent may be made from a single piece and may have a porous or corrugated surface on one or both the exterior and interior sides. The stent may have holes or groves completely through it to provide the vessel with nutrients.

During fabrication, the stent can be annealed around an appropriately sized rod or mandrel, to increase radial strength as well as dimensional and geometric stability.

One or more layers of the stent may be braided, woven, wrapped or other pattern. The stent may have layers consisting of polymer, metal or a combination for surface texture and strength.

A method of use for the stent includes placing it around the exterior of the lumen and closing it into its cylindrical shape. The lumen is pushed against the interior of the stent where it comes into contact with the adhesive and sticks to the interior of the stent thereby enlarging and opening the lumen. The lumen is expanded to push against the stent by an angioplasty balloon or other similar device. It should be noted that the stent need not provide complete circumferential contact. The design could offer sufficient reinforcement to the vessel with as little as 40% coverage and possibly less.

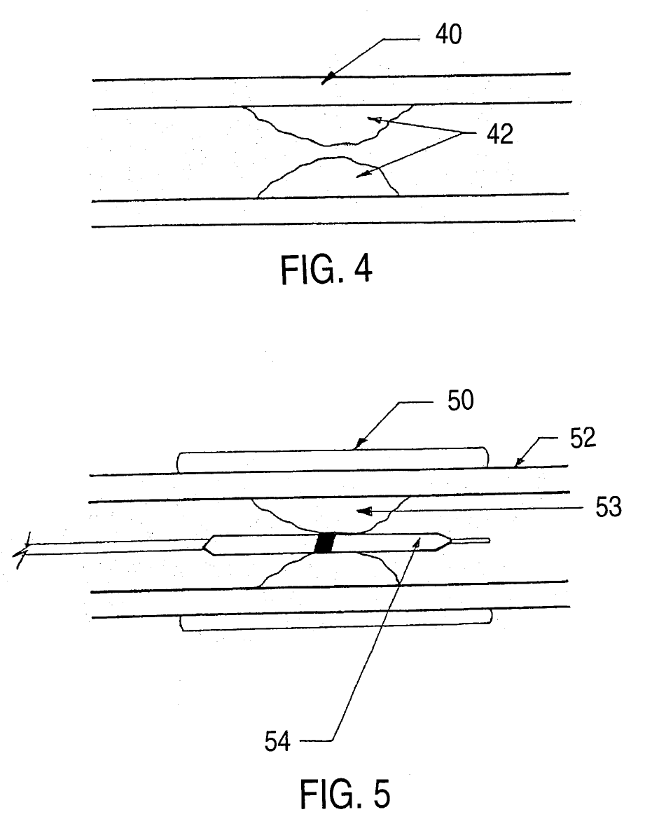

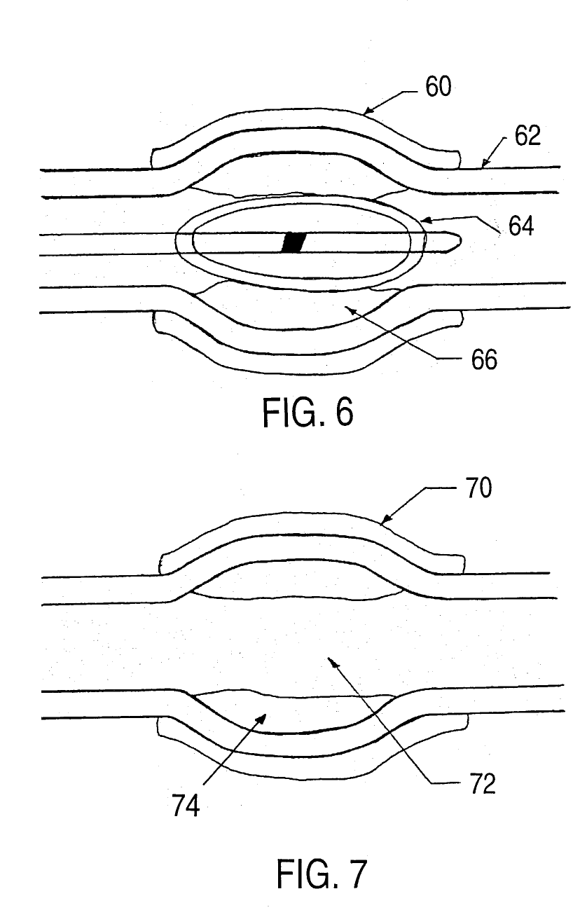

The stent may be placed around a target lumen such as the one in FIG. 4, having a lesion 42, and ratcheted or compressed down to contact the exterior of the lumen wall. The contact is sufficient when the lumen becomes secure to the stent by the barbs or adhesive or other securing means. Alternatively, a PTCA balloon catheter depicted as 54 in FIG. 5 may be advanced within the lumen 52 into place with the balloon positioned at the site of the external stent 50. As shown in FIG. 6, the balloon 64 is contact with a lesion 66 (also depicted as 53 in FIG. 5) then inflated causing the lumen to expand and come into contact with the interior side of the stent bonding the lumen to the stent. After the stent 70 is secured, the balloon is then deflated and the catheter withdrawn, resulting in the lumen having a larger diameter 72 as the narrowing caused by the lesion 74 in FIG. 7 has been widened.

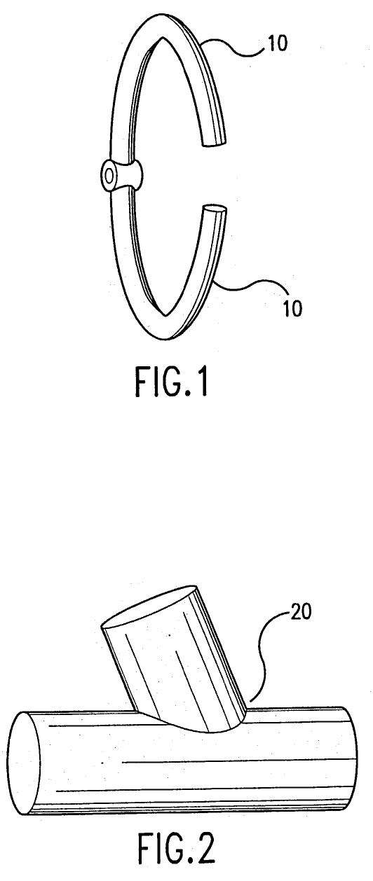

In an alternative embodiment and as shown in FIG. 2, the stent may be bifurcated or more to provide for coverage of bifurcated lumens.

Accordingly, it should be readily appreciated that the exterior stent and the uses of the present invention has many practical applications. Additionally, although the preferred embodiment has been illustrated and described, it will be obvious to those skilled in the art that various modifications can be made without departing from the spirit and scope of this invention. Such modifications are to be considered as included in the following claims unless the claims expressly recite differently.

What is claimed:

1. An exterior stent comprising:
a support layer having an inner diameter and an outer diameter, and
a securing element on the inner diameter of the support layer wherein said securing element in is contact with an exterior diameter of a body lumen.

2. A stent as in claim 1 wherein:
the support layer has an inner diameter greater than the exterior diameter of the lumen.

3. A stent as in claim 1 wherein:
the support layer comprises biologically inert material.

4. A stent as in claim 3 wherein:
the inert material is a shape-memory material.

5. A stent as in claim 3 wherein:
the inert material is PTFE.

6. A stent as in claim 3 wherein:
the inert material is Dacron.

7. A stent as in claim 3 wherein:
the support layer further comprises a biologically active material.

8. A stent as in claim 7 wherein:
the active material is a drug-releasing coating on a surface of the stent that permits timed or prolonged pharmacological activity.

9. A stent as in claim 1 wherein:
the support layer comprises resorbable material.

10. A stent as in claim 9 wherein:
the support layer further comprises a biologically active material.

11. A stent as-in claim 10 wherein:
the support layer is shape-memory material.

12. A stent as in claim 1 wherein:
the support layer is porous for providing nutrients or irrigation to the lumen.

13. A stent as in claim 1 wherein:
the support layer comprises a braided material.

14. A stent as in claim 1 wherein:
the support layer is a single unified member.

15. A stent as in claim 1 wherein:
the support layer comprises at least two members flexibly joined together.

16. A stent as in claim 15 wherein:
the members are joined by a hinge.

17. A stent as in claim 1 wherein:
the support layer is bifurcated.

18. A stent as in claim 1 wherein:
the support layer comprises a radioactive element for delivering radiation directly to the lumen.

19. A stent as in claim 18 wherein:
the support layer further comprises a biologically active material.

20. A stent as in claim 1 wherein:
the securing element is a barb.

21. A stent as in claim 1 wherein:
the securing element is a hook.

22. A stent as in claim 1 wherein:
the securing element is an adhesive.

23. A stent as in claim 22 wherein:
the adhesive is biologically inert.

24. A stent as in claim 22 wherein:
the adhesive requires curing.

25. A stent as in claim 1 wherein:
the securing element is a suture.

26. A stent as in claim 1 wherein:
the securing element are locks that close the stent tightly onto the lumen to prevent it from slipping but not to restrict the lumen.

27. A stent as in claim 1 wherein:
the support layer covers less than the entire circumference of the lumen.

28. A stent as in claim 1 further comprising:
a reinforcing layer for strengthening the support layer.

29. A stent as in claim 28 wherein:
the reinforcing layer comprises a braided material.

30. A method of supporting a body lumen comprising:
placing a support around the exterior of a body lumen; and securing the support to the lumen.

31. A method of support as in claim 30 wherein:
the support covers less than the total circumference of the lumen.

32. A method of support as in claim 30 wherein:
the support comprises a biologically inert material.

33. A method of support as in claim 32 wherein:
the support further comprises a shape-memory material.

34. A method of support as in claim 30 wherein:
the support comprises a biologically active material.

35. A method of support as in claim 30 wherein:
the support comprises resorbable material.

36. A method of support as in claim 30 wherein:
the support comprises a radioactive element for delivering radiation directly to the lumen.

37. A method as in claim 30 wherein:
the support is porous.

38. A method of support as in claim 30 wherein:
the support is a single unified member.

39. A method of support as in claim 30 wherein:
the support comprises at least two members flexibly joined together.

40. A method of support as in claim 30 wherein:
the support is bifurcated.

41. A method of support as in claim 30 wherein:
the support is secured by a barb.

42. A method of support as in claim 30 wherein:
the support is secured by a hook.

43. A method of support as in claim 30 wherein:
the support is secured by an adhesive.

44. A method of support as in claim 30 wherein:
the support comprises a braided material.

45. A method of support as in claim 30 wherein:
the support is substantially composed of resorbable material.

46. A method of support as in claim 30 wherein:
the support is porous.

47. A method of support as in claim 30 wherein:
the support comprises at least two members flexibly joined together.

48. A method of support as in claim 30 wherein:
the support is secured by sutures.

49. A method of support as in claim 30 wherein:
the support locks onto the lumen to prevent it from slipping.

50. A method of support as in claim 30 further comprising:
a reinforcing layer for strengthening the support.

51. A method for implanting a prosthesis to an exterior of a body lumen comprising:
a) providing for a stent as described in claim 1;
b) inserting the stent around a desired location on the exterior of the lumen; and
c) providing for controllable contraction of the prosthesis at the desired location by exerting a force upon the prosthesis to deform it such that it contacts the lumen sufficiently to secure it to the lumen.

52. A method for implanting a prosthesis to an exterior of a body lumen comprising:
a) providing for a stent as described in claim 1;
b) inserting the stent around a desired location on the exterior of the lumen; and
c) providing for controlled expansion of the lumen such that it contacts the stent sufficiently to secure it to the lumen.
